(19) United States
(12) Patent Application Publication
(10) Pub. No.: US 2004/0253298 A1
(43) Pub. Date: Dec. 16, 2004

(54) Body dressing

(76) Inventor: Qi Yu, Rosemead, CA (US)

Correspondence Address: Raymond Y. Chan, Suite 128, 108 N. Ynez Ave., Monterey Park, CA, 91754, US

(21) Appl. No.: 10/868,566

(22) Filed: Jun. 14, 2004

Related U.S. Application Data
(60) Provisional application No. 60/478,158, filed on Jun. 13, 2003.

(51) Int. Cl. A61N 1/30; A61L 15/00

(52) U.S. Cl. 424/445

(57) ABSTRACT

A body dressing includes a permeating fluid adapted for applying on a skin surface of treating tissues of a user and an ion carrier which is mixed with the permeating fluid containing positively charged ions for depolarizing negative ions accumulated under the skin surface of the treating tissue around sweat glands of the user. The permeating fluid is adapted for permeating into the treating tissues through the skin surface so as to provide an ion passway between the skin surface in contact with the permeating fluid and the treating tissues so as to enhance the depolarization of the positively charged ions of the ion carrier with the negative ions around the treating tissues of the sweat glands of the user.

### CROSS REFERENCE OF RELATED APPLICATION

This is a Regular Application of a non-provisional application, application No. 60/478,158, filed Jun. 13, 2003.

### BACKGROUND OF THE PRESENT INVENTION

1. Field of Invention

The present invention relates to a therapeutic application, and more particularly to a body dressing which provides positively charged ions for depolarizing a negatively charged muscular tissues around sweat glands under the human skin, so as to efficiently cure the skin disease such as armpits odor, pimple, leuconychia, and athlete's foot.

2. Description of Related Arts

Nowadays, people are suffering many skin diseases, especially armpits odor. Unfortunately, the treatments of armpits odor are either applying a stronger smell to cover the odor or lowering down the rate of sweat. However, such treatments cannot cure the armpits odor.

In order to cure the armpits odor, human metabolism must be recognized. Human body is considered as a system that is able to be self-regulated the body temperature such that the human body excretes moisture from the sweat glands to reduce the body temperature. When fungus grows along the sweat gland which is located under the human skin approximate 0.5 to 1.5 centimeter, the fungus will be stuck in the sweat gland to release a smell and cause the skin diseases, such as armpits odor, pimple, leuconychia, or athlete's foot. Practically, the fungus can only grow under a condition that the sweat gland has a negative electric signal. In other words, the negative ions around the sweat gland will enhance the growth of the fungus.

Since such treatments of armpits odor are usually applied on the human skin, the treatment cannot permeate through the human skin to the sweat gland in order to kill the fungus comprehensively. Besides, the human skin, especially sensitive skin, may have allergy since the treatments are composed of many chemical substances.

### SUMMARY OF THE PRESENT INVENTION

A main object of the present invention is to provide a body dressing which comprises a dressing layer containing positively charged ions for depolarizing the negatively charged ions in a treating tissue around the sweat gland, wherein the dressing layer further comprises a permeating fluid to create an ion passway between the skin in contact with the dressing layer of the body dressing and the treating tissue so as to enhance the depolarization of the positively charged ions of the dressing layer with the negatively charged ions in the treating tissue of the sweat gland.

Another object of the present invention is to provide a body dressing wherein the carcasses of the fungus are excreted out from the treating tissue through the sweat gland so as to comprehensively cure the skin disease.

Another object of the present invention is to provide a body dressing wherein neither magnetic field nor chemical substance is used and permeated into the human skin, so that it is safe to use with allergy free.

Accordingly, in order to accomplish the above objects, the present invention provides a body dressing, comprising:

a permeating fluid adapted for applying on a skin surface of treating tissues of a user; and

an ion carrier which is mixed with the permeating fluid containing positively charged ions for depolarizing negative ions accumulated under the skin surface of the treating tissue around sweat glands of the user;

wherein the permeating fluid is adapted for permeating into the treating tissues through the skin surface so as to provide an ion passway between the skin surface in contact with the permeating fluid and the treating tissues so as to enhance the depolarization of the positively charged ions of the ion carrier with the negative ions around the treating tissues of the sweat glands of the user.

The present invention also provides a process of making the body dressing, which comprises the steps of:

(a) grinding a metallic element into a fine powder form; and

(b) mixing a permeating fluid with the metallic element to form the body dressing, wherein the ratio of the metallic element and the permeating fluid is 1:9 by weight.

### BRIEF DESCRIPTION OF THE DRAWINGS

FIGS. 1A and 1B illustrate a body dressing applied on a human skin according to a preferred embodiment of the present invention.

FIG. 2 is a flow chart illustrating the manufacturing process of the body dressing according to the above preferred embodiment of the present invention.

### DETAILED DESCRIPTION OF THE PREFERRED EMBODIMENT

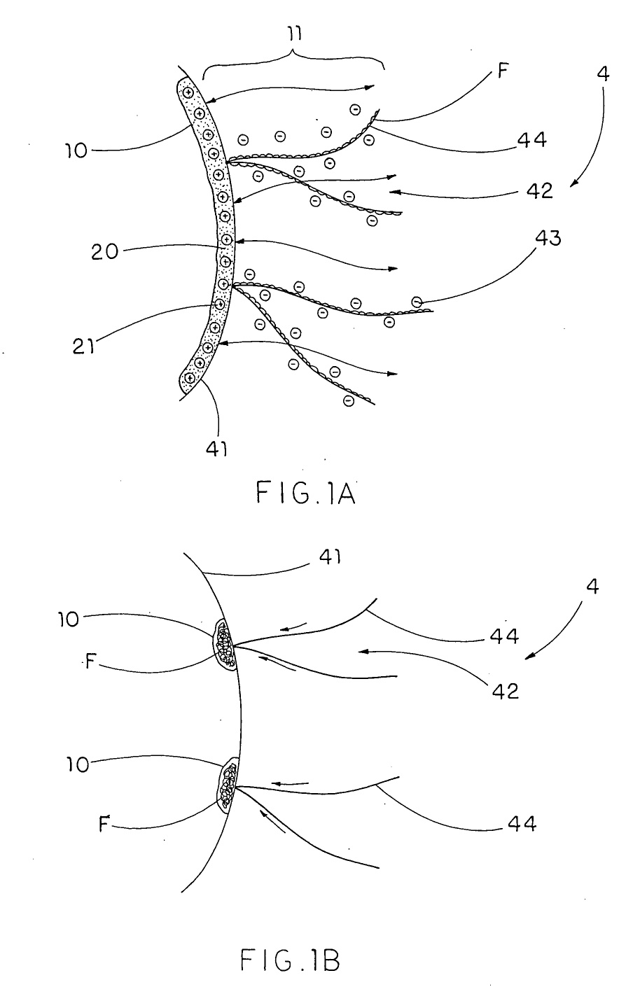

Referring to FIG. 1 of the drawings, a body dressing according to a preferred embodiment of the present invention is illustrated, wherein the body dressing comprises a permeating fluid 10 adapted for applying on a skin surface 41 of treating tissues 42 of a user 4 and an ion carrier 20 which is mixed the permeating fluid 10 containing positively charged ions 21 for depolarizing negative ions accumulate under the skin surface 41 of the treating tissue around sweat glands 44 of the user 4.

The permeating fluid 10 is adapted for permeating into the treating tissues 42 through the skin surface so as to provide an ion passway 11 between the skin surface 41 in contact with the permeating fluid 10 and the treating tissues so as to enhance the depolarization of the positively charged ions 21 of the ion carrier 20 with the negative ions 43 around the treating tissues 42 of the sweat gland 44 of the user.

The ion carrier 20 which is mixed with the permeating fluid 10 is positive ion carrier. According to the preferred embodiment, the ion carrier 20 is made of powder form oxidized copper which is positively charged to form the positive ions 21. It is worth to mention that other metallic elements such as zinc, gold, or pure sliver can be used to substitute the copper to make the fine powder form positive ion carrier.

Billions of positively charged ions are loaded in the body dressing of the present invention, which is hundred or even thousand times more than the negative ions 43 generally existed at the tissues to be treated. However, the body skin and muscle between the treating tissues 42 and the body dressing is an alterative obstacle of the depolarization of positive ions 21 and the negative ions 43.

Accordingly, an essential feature of the present invention is to include a kind of ion carrier, i.e. permeating fluid 10, to provide a passway between the skin surface 41 and the treating tissues 42 for the positively charged ions 21. The permeating fluid 10 is Jojoba oil that adapted for permeating through the skin surface 41 approximately 3 centimeters thereunder.

It is well known that the Jojoba oil is extracted from Jojoba wherein the Jojoba oil has only one alcohol group and is a straight chain molecule. Therefore, it is not subject to oxidizing and in fact is an anti-oxidant and will never become rancid. Jojoba oil is a natural mimic of the oil secreted by human skin so it can be used to protect and lubricate skin and hair. It is soothing, stops multitudinous skin problems and protects against premature aging and wrinkling of the skin caused by exposure to ultra violet radiation.

Furthermore, since the Jojoba oil of the permeating fluid 10 is a natural resource and will never become rancid, the Jojoba oil of the permeating fluid 10 is absolutely safe to use and allergy free.

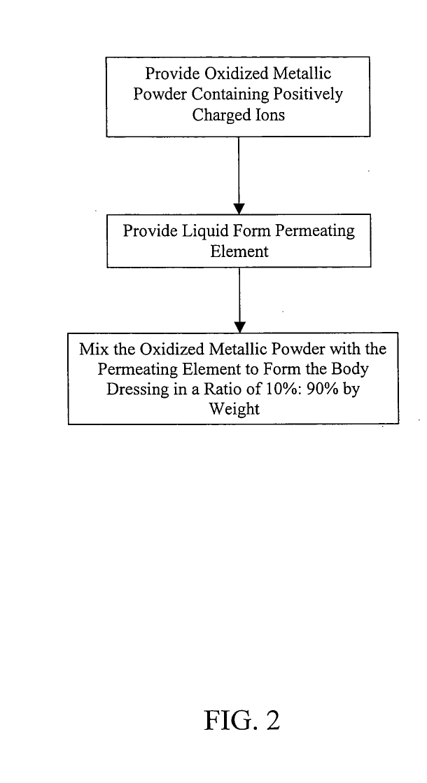

As shown in FIG. 2A, since the sweat gland 44 is located under the skin surface 41 of the user 4 approximate 0.5 to 1.5 centimeter, the Jojoba oil of the permeating fluid 10 is capable of penetrating through the skin surface 41 of the user 4 to reach a position where the sweat glands 44 are located. Thus, when the permeating fluid 10 permeate into the human skin and the tissues, the ion passway 32 is formed between the skin surface 41 and the treating tissues 42 to enhance the communication of the positively charged ions 21 and the negative ions 43 at the treating tissues 42.

In order to kill the fungus F in the sweat gland 44, the body dressing is arranged to apply on the human skin where the fungus F is located within the sweat gland 44. Due to the present of the ion passway 11 created by the permeating fluid 10, the depolarization rapidly proceeds via the ion passway 11 that the smaller amounted of negative ions 43 generated at the treating tissues 42 around the sweat gland 44 will be rapidly neutralized by the numerous quantity of positively charged ions 21. When the negative ions 43 at the treating tissues 42 is depolarized, tissue cells become normal and no electric charge is around the sweat gland 44. Therefore, the fungus F will be inevitably killed in the sweat gland 44 under the neutralized condition of the tissue cells therearound.

Moreover, when the fungus F is killed and remained in the sweat gland 44, the carcasses of the fungus F will also be excreted out of the human skin along the sweat gland 44 with the permeating fluid 10 through the pores during sweating, as shown in FIG. 2B. Therefore, the carcasses of the fungus F will be cleaned up to provide a hygienic condition of the sweat gland 44 and to prevent the odor produced by the carcasses of the fungus F.

Alternatively, the Jojoba oil of the permeating fluid 10 can be substituted by the Sperm whale oil which is also adapted for permeating through the human skin to a position where the sweat glands 44 are located, so as to form the ion passway 11 between the skin surface 41 in contact with the permeating fluid 10 and the treating tissues 42.

Referring to FIG. 3, the body dressing of the present invention is produced by the following steps.

(a) Provide an oxidized metallic powder containing positively charged ions 21 by grinding a predetermined amounted of oxidized metallic element into a fine powder. It is preferred that the oxidized metallic powder is as fine as 1000 meshes.

(b) Provide a permeating fluid which is adapted for is adapted for permeating through a skin surface of a user.

(c) Evenly mix the permeating fluid 10 with the oxidized metallic powder to form the body dressing.

In other words, according to the preferred embodiment as shown in FIGS. 1 to 3, the body dressing is made of the positively charged ions 21 and the permeating fluid 10.

In step (c), the oxidized metallic powder is mixed with the permeating fluid 10 in a ratio of 10%:90% by weight. Accordingly, the body dressing of the present invention is formed liquid form in such a manner that the body dressing is adapted for applying on the skin surface 41 of the treating tissues 42 of the user 4 via a spraying device or a sponge means. Alternatively, the body dressing is adapted to be embodied as a cream form for directly applying on the skin surface 41 of the treating tissues 42 of the user 4.

According to the preferred embodiment, the body dressing further comprises a medical element 30 mixed therewith wherein the medical element 30 can be a medicine such as muscle stress reliever, Chinese medical herb or lotion adapted for permeating under the human skin to enhance the treatment effect. It is worth to mention that when the medical element 30 is in a powdered form, the medical element 30 is preferably mixed with the oxidized metallic powder in step (1) to form a mixing powder and then mixed with the permeating fluid 10. When the medical element 30 is in a liquid form, the medical element 30 is preferably mixed with the permeating fluid 10 before mixing with the oxidized metallic powder. However, it should not be limited the procedure of mixing the medical element 30 with the oxidized metallic powder and the permeating fluid 10 in order to bring out the own feature of the medical element 30.

What is claimed is:

1. A body dressing, comprising:
a permeating fluid adapted for applying on a skin surface of treating tissues of a user; and
an ion carrier which is mixed with said permeating fluid containing positively charged ions for depolarizing negative ions accumulated under said skin surface of said treating tissue around sweat glands of said user;
wherein said permeating fluid is adapted for permeating into said treating tissues through said skin surface so as to provide an ion passway between said skin surface in contact with said permeating fluid and said treating tissues so as to enhance said depolarization of said positively charged ions of said ion carrier with said negative ions around said treating tissues of said sweat glands of said user.

2. The body dressing, as recited in claim 1, wherein said ion carrier is made of a powder form oxidized metallic element which is positively charged to load with said positively charged ions.

3. The body dressing, as recited in claim 1, wherein said permeating fluid is Jojoba oil which is adapted for permeating through said skin surface to a position where said sweat glands are located.

4. The body dressing, as recited in claim 2, wherein said permeating fluid is Jojoba oil which is adapted for permeating through said skin surface to a position where said sweat glands are located.

5. The body dressing, as recited in claim 1, wherein said permeating fluid is sperm whale oil which is adapted for permeating through said skin surface to a position where said sweat glands are located.

6. The body dressing, as recited in claim 2, wherein said permeating fluid is sperm whale oil which is adapted for permeating through said skin surface to a position where said sweat glands are located.

7. The body dressing, as recited in claim 1, wherein 90% by weight of said permeating fluid is mixed with 10% by weight of said ion carrier.

8. The body dressing, as recited in claim 2, wherein 90% by weight of said permeating fluid is mixed with 10% by weight of said ion carrier.

9. The body dressing, as recited in claim 4, wherein 90% by weight of said permeating fluid is mixed with 10% by weight of said ion carrier.

10. The body dressing, as recited in claim 6, wherein 90% by weight of said permeating fluid is mixed with 10% by weight of said ion carrier.

11. A process of making a body dressing, comprising the steps of:
(a) providing an oxidized metallic powder containing positively charged ions;
(b) providing a permeating fluid which is adapted for permeating through a skin surface of a user; and
(c) mixing said oxidized metallic powder with said permeating fluid to form said body dressing.

12. The process as recited in claim 11 wherein, in step (c), said oxidized metallic powder is mixed with said permeating fluid in a ratio of 10%:90% by weight.

13. The process, as recited in claim 11, wherein said permeating fluid is Jojoba oil which is adapted for permeating through said skin surface to a position where said sweat glands are located.

14. The process, as recited in claim 12, wherein said permeating fluid is Jojoba oil which is adapted for permeating through said skin surface to a position where said sweat glands are located.

15. The process, as recited in claim 11, wherein said permeating fluid is sperm whale oil which is adapted for permeating through said skin surface to a position where said sweat glands are located.

16. The process, as recited in claim 12, wherein said permeating fluid is sperm whale oil which is adapted for permeating through said skin surface to a position where said sweat glands are located.

17. The process as recited in claim 13, in step (a), further comprises a step of mixing a powder form medical element with said oxidized metallic powder.

18. The process as recited in claim 15, in step (a), further comprises a step of mixing a powder form medical element with said oxidized metallic powder.

19. The process as recited in claim 13, in step (b), further comprises a step of mixing a liquid form medical element with said permeating fluid.

20. The process as recited in claim 15, in step (b), further comprises a step of mixing a liquid form medical element with said permeating fluid.
